(19) United States
(12) Patent Application Publication
(10) Pub. No.: US 2020/0311388 A1
(43) Pub. Date: Oct. 1, 2020

(54) HUMAN BODY PHYSIOLOGICAL PARAMETER MONITORING METHOD BASED ON FACE RECOGNITION FOR WORKSTATION

(71) Applicant: LOCTEK ERGONOMIC TECHNOLOGY CORP., Ningbo (CN)

(72) Inventor: LEHONG XIANG, NINGBO (CN)

(73) Assignee: LOCTEK ERGONOMIC TECHNOLOGY CORP.

(21) Appl. No.: 16/833,253

(22) Filed: Mar. 27, 2020

(30) Foreign Application Priority Data
Mar. 27, 2019 (CN) 201910236620.8

(51) Int. Cl. G06K 9/00; A61B 5/02; A61B 5/00; G06F 17/16

(52) U.S. Cl. CPC G06K 9/00288; A61B 5/02; G06F 17/16; A61B 5/0013; A61B 5/441

(57) ABSTRACT

The present disclosure is a human body physiological parameter monitoring method based on face recognition for a workstation, and the method is based on a human physiological parameter monitoring system. The human physiological parameter monitoring system has a backstage server, at least one image acquisition device arranged in the workstation; the image acquisition device is communicatively connected with the backstage server and the method has the following steps:(1) continuous image sampling is carried out by the image acquisition device, and uploaded to the backstage server; when an image acquisition device detects the presence of a person, it proceeds to step (2); (2) the backstage server compares the person detected in step (1) with a pre-stored registered person sample on the backstage server through a face recognition algorithm. The method disclosed has the advantage of greatly improved detection efficiency and accuracy.

### CROSS-REFERENCE TO RELATED APPLICATIONS

This application claims priority benefits to Chinese Patent Applications No. 201910236620.8, filed on Mar. 27, 2019. The contents of all of the aforementioned applications, including any intervening amendments thereto, are incorporated herein by reference.

### TECHNICAL FIELD

The present disclosure relates to the technical field of image recognition and tracking, in particular to a human body physiological parameter monitoring method based on face recognition for a workstation.

### BACKGROUND

Face recognition technology has gained more and more importance in social life, and presently, a face recognition technology has been applied to the monitoring of human physiological parameters. For example, the Chinese patent application with publication number CN104182725 discloses a face recognition and tracking method based on non-contact human physiological parameter measurement, and the method comprises the following steps: step (1), detecting a face in a frame of an acquired image or video stream and separating the face from the background; step (2), performing feature extraction on the captured face image, and registering the extracted face feature; step (3), detecting whether a registered face exists within the shooting range of the camera, if the face exists, when it's captured on the camera when moving within the range, the face is automatically tracked and saved; if the face does not exist, return to step (2) to register it and update the registration information database. According to the face recognition and tracking method based on non-contact human physiological parameter measurement, when a person is detected to be a registered object, his/her face is automatically tracked and automatically saved; when a person is detected as an unregistered object, his/her face is registered, and the registration information database is updated.

However, the existing human body physiological parameter monitoring method based on face recognition monitors physiological parameters regardless of whether it is a registered object or an unregistered object, thus the method is suitable for occasions such as airports and train stations which are not directed to a specific group of people. A worker in the workstation is specific, so that when the existing human physiological parameter monitoring method based on face recognition is applied to the workstation, it will obviously monitor unnecessary people, leading to a greatly reduced detection efficiency and affected detection accuracy.

### SUMMARY

One objective of the present disclosure is to overcome the shortcomings of the prior arts by providing a human body physiological parameter monitoring method based on face recognition for a workstation with greatly improved detection efficiency and accuracy.

The technical solution is that the human body physiological parameter monitoring method based on face recognition for a workstation, and the method is based on a human physiological parameter monitoring system; the said human physiological parameter monitoring system comprises a backstage server, at least one image acquisition device arranged in the workstation; the image acquisition device is communicatively connected with the backstage server and the method comprises the following steps:

(1) continuous image sampling is carried out by the image acquisition device, and uploaded to the backstage server; when an image acquisition device detects the presence of a person, it proceeds to step (2);

(2) the backstage server compares the person detected in step (1) with a pre-stored registered person sample on the backstage server through a face recognition algorithm;

if the current person is a registered object, the backstage server stores the person's current physiological parameter information into a database of this person for subsequent analysis;

If the current person is an unregistered object, the person is ignored. With the above method, the present invention has the following advantages:

the human body physiological parameter monitoring method based on face recognition for a workstation of the present invention only monitors human body physiological parameter of registered objects; the registered objects are personnel associated with the workstation, which not only meets the monitoring requirements of the workstation, but also saves time in the detection of irrelevant personnel, thus the detection efficiency is greatly improved, and the real-time performance better; besides, ignoring unregistered objects and only aiming at registered objects, the target is more precise, and the detection content is more simplified since less irrelevant interference is received, which in turn makes it more conducive to improving the accuracy of detection.

Preferably, the present invention further comprises a user terminal device communicatively connected with the backstage server, and the current working status of the system comprises a registering status and a monitoring status; the current working status of the system is initialized to the monitoring status upon power-on, and the user terminal device communicates with the backstage server through a pre-agreed communication mode to enter the registering status; in the said step (1), before the continuous image sampling is performed by the image acquisition device in the workstation and uploaded to the background server, the backstage server judges whether the current working status of the system is a registering status or a monitoring status, and only when the current working status of the system is in the monitoring status, continuous image sampling is performed by the image acquisition device in the workstation and uploaded to the backstage server, otherwise, it proceeds to step (3): the current working status of the system is the registering status, and the head portrait of the person is stored in the database of the person. This setting enables the system to have both registering status and monitoring status, which ensures a good controllability on the basis of meeting user's operational requirements and flexibility. Preferably, the user terminal device is a mobile phone; when the current working status of the system is a registering status, the head portrait is collected by the camera of the mobile phone and uploaded to the backstage server, and the backstage server saves the head portrait of the person to the database of the person. Registering with a mobile phone makes the head portrait collection very convenient.

Preferably, the physiological parameter information of a person includes heart rate and blood flow, and the heart rate and blood flow are obtained by analyzing the continuous frame images acquired by the image acquisition device. Under this setting, the image acquisition device is not only used for recognizing human faces, but also used for acquiring physiological parameter information, without the need to additionally arrange a physiological parameter detection equipment, which greatly saves the cost.

Preferably, it comprises the following steps to obtain the heart rate and blood flow information through analyzing the continuous frame images acquired by the image acquisition device:

S1, capture continuous frame images of a person;

S2, extract the collected RGB information of the skin area of each frame image, and then obtain three matrices based on the information of the three channels of the extracted RGB;

S3, perform a dimension reduction on the three matrices obtained in each frame image in step S2, and three new matrices are obtained in each frame image;

S4, average the three new matrices obtained in each frame image in step S3, and respectively obtain an average value of each new matrix of each frame image; then use “time” as the abscissa, and “the average value of the new matrix of the R channel” as the vertical ordinate to obtain a first waveform diagram; use “time” as the abscissa, and “the average value of the new matrix of the G channel” as the vertical ordinate to obtain a second waveform diagram; use “time” as the abscissa, and “the average value of the new matrix of the B channel” as the vertical ordinate to obtain a third waveform diagram;

S5, filter the three waveform diagrams obtained in step S4 through a filter;

S6, combine the three waveform diagrams filtered in step S5;

S7, extract the periodic signal as a heart rate signal and the envelope signal as a blood flow signal in the waveform diagram combined in step S6.

According to the method, the heart rate and blood flow information can be accurately acquired, and the dimension reduction process and average calculation reduce the amount of computation, making the detection of physiological information faster.

Preferably, step S1 is acquiring a face image, and step S2 is extracting facial skin region. The facial skin image is more convenient and accurate to be identified compared with skin image in other regions.

Preferably, in step S4, the three new matrices need to be subjected to weighted average calculation; the weighted average calculation method is: sequentially arranging the difference values of frames before and after the dimension reduction matrix, filtering out pixels whose absolute value of change is greater than a set threshold value, calculating the average value of the remaining pixel values and this value is the average value of the channel of the current frame. By means of a weighted average algorithm, the accuracy of the identification is higher.

Preferably, the dimension reduction in step S3 refers to smoothing and downsizing the matrix. By smoothing and downsizing, less calculation amount is required, and the recognition efficiency is relatively higher.

Preferably, before acquisition in step S1, image brightness detection is further required, and if the detected image brightness is insufficient, exposure compensation is required until the detected image brightness meets the standard. The image brightness detection is performed before the image is acquired, which ensures sufficient brightness for the collected recognition image, thereby improving the accuracy of subsequent recognition and judgments.

### BRIEF DESCRIPTION OF THE DRAWINGS

FIG. 1 is a flowchart of a human physiological parameter monitoring method based on face recognition applied to a workstation of the present invention;

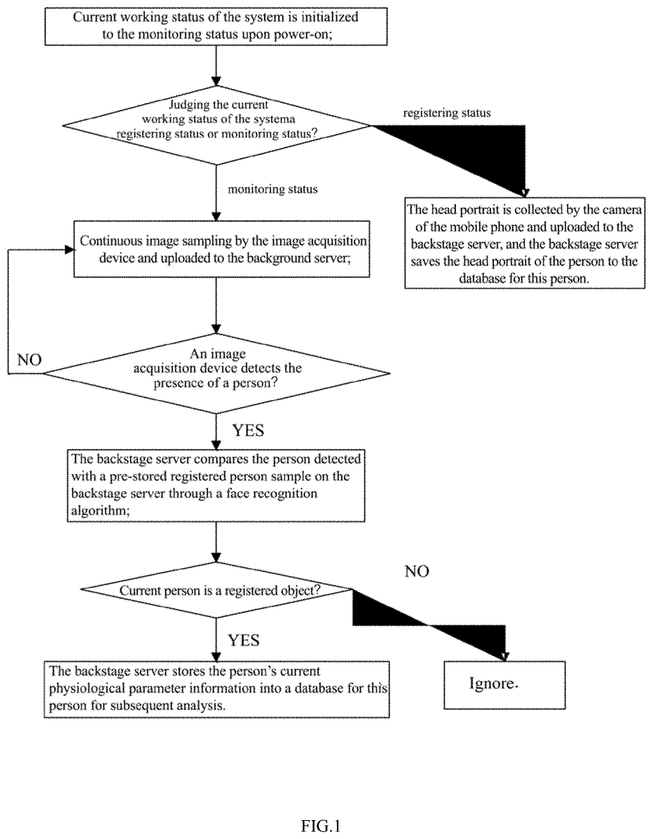

FIG. 2 is a signal schematic diagram after single-channel weighted average of continuous frame images of the present invention;

FIG. 3 is a schematic diagram of the filtered signals of FIG. 2;

FIG. 4 is a signal schematic diagram of time-domain information after the combination of the three-channels of continuous frame images of the present invention.

### DETAILED DESCRIPTION OF THE EMBODIMENTS

The invention is further described below with reference to the accompanying drawings.

### Embodiment

A human body physiological parameter monitoring method based on face recognition for a workstation, and the method is based on a human physiological parameter monitoring system; the said human physiological parameter monitoring system comprises a backstage server, an user terminal device, at least one image acquisition device arranged in the workstation; the user terminal device and the image acquisition device are both communicatively connected with the backstage server; the image acquisition device can be arranged on a lifting platform or a heightening desk or a lifting table or arranged on an accessory of the lifting platform or the heightening desk or the lifting table; the user terminal device is a mobile phone; and the current working status of the system comprises a registering status and a monitoring status; the method includes the following steps :

(1) the current working status of the system is initialized to the monitoring status upon power-on;

(2) judge the current working status of the system: monitoring status or registering status; the mobile phone communicates with the backstage server through a pre-agreed communication mode to enter the registering status; the pre-agreed communication mode may use the existing technology, such as setting a password; If the current working status of the system is in the monitoring status, proceed to step (3);

If the current working status of the system is in the registering status, the head portrait of the person is collected by the camera of the mobile phone and uploaded to the backstage server, the backstage server stores the head portrait of the person in the database of this person;

(3) continuous image sampling is carried out by the image acquisition device and uploaded to the backstage server, and when an image acquisition device detects that a person appears, proceed to step (4);

(4) the backstage server compares the person detected in step (3) with a pre-stored registered person sample on the backstage server through a face recognition algorithm;

if the current person is a registered object, the backstage server stores the person's current physiological parameter information into a database of this person for subsequent analysis;

If the current person is an unregistered object, the current person is ignored.

Preferably, the physiological parameter information of a person includes heart rate and blood flow, and the heart rate and blood flow are obtained by analyzing the continuous frame images acquired by the image acquisition device. Under this setting, the image acquisition device is not only used for recognizing human faces, but also used for acquiring physiological parameter information, without the need to additionally arrange a physiological parameter detection equipment, which greatly saves the cost.

Preferably, it comprises the following steps to obtain the heart rate and blood flow information through analyzing the continuous frame images acquired by the image acquisition device:

Firstly an exposure compensation adjustment is required: the system corrects and locks the exposure value during initialization, and the selection of the exposure value includes, but is not limited to, the following method: comparing and adjusting the numerical histogram with the empirical histogram of the whole picture; if the histogram is relatively dark, improving the overall numerical brightness; adjusting by detecting the brightness of the facial region; adjusting the exposure compensation by detecting the comparison between the facial region and other regions; in the algorithm detection process, the change of the ambient light can be tracked by monitoring the background brightness value, and the change of the ambient light can be used as a compensation feedback to the face value (especially when a continuous moving object appears in the background).

In some embodiment, the image histogram values can be provided to the user as a feedback indicating whether the office light is appropriate: e. g., when the ambient light is detected to be weak, remind the user to increase a light source and/or the light source intensity.

Then, continuous frame images of the human face are acquired through the camera;

Next, by running the face detection algorithm, a color matrix corresponding to the area range of the three channels of RGB is obtained;

After the facial region is locked, the values of the three channels of the RGB of the image are respectively extracted into a matrix of M*N, and M is the width of the facial region, N is the height (the value of M and N is variable according to the distance of the person, in real practice, the system can perform image correction through the value change of M and N, and M can be set to 640 and N to 480 in present invention).

Furthermore, assuming that the variations of the image between a frame and the next frame are not severe, i.e., there is no strong displacement of the measured object, the average value of a certain small area (such as 5*5 gaussian kernel) can be regarded as a single pixel value after filtering to ensure that the algorithm will not be interfered by the noise generated by the external environment or the hardware of the capture device, so that the blood pulsation information can be obtained by comparing the changes of the same pixel point.

Thereafter, in order to reduce the computational intensity and noise interference, the color matrix needs to be processed with dimension reduction (Gaussian pyramid or Gaussian blur and other moving average methods and the like) to obtain three relatively small matrices, that is, to mainly reduce the size, such as a matrix of 640*480 is reduced to 160*120, and then a further weighted average of the signals per second of each matrix is needed; in this embodiment, the method of weighted average is: sequentially arranging difference values of the frames before and after the dimension reduction matrix, a certain percentage of pixel points with relatively large absolute values are filtered out, and calculate the average value of the remaining pixel values, which is the average value of the channel of the current frame.

In addition, due to the movement and the facial motion of the measured object, the values of the corresponding pixels in continuous frames can generate a jump, and if all the pixel points are weighted, a jump of the signal baseline can be caused. In the algorithmic framework herein, the computation of outlier removal is introduced: take the absolute value of the difference between the corresponding pixel points of continuous frames, compare the distribution of absolute values in the region with an empirical template (assuming that the value is subject to a normal distribution ideally), and find fitting parameters of the empirical distribution through distribution fitting; the out-of-region portion is treated as outlier.

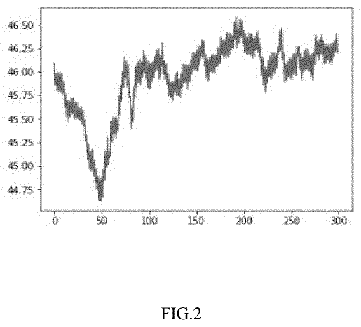

As shown in FIG. 2, a 10-second original weighted average signal of a single channel (the camera uses a frequency of 30FPS). Due to the instability of the facial region, we see that the background noise of the original average signal is large.

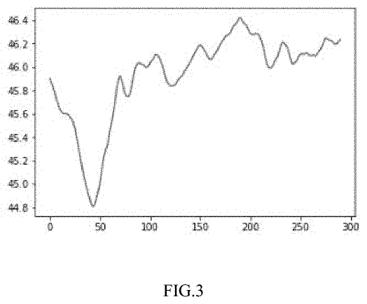

Considering the basic noise of the hardware and the jitter of the tracking of the facial region, a low-pass filtering is performed on FIG. 2, considering that the normal heart rate range is 0.5-3 Hz, we set the corresponding band-pass filter for filtering; FIG. 3 shows the effect after filtering, or, if the breath detection is in the range of 0.1-0.5 Hz, the corresponding band-pass filter can also be set for filtering;

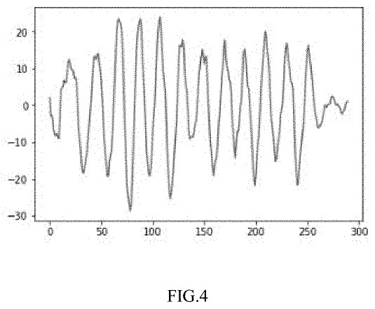

Due to the difference between the reception intensity of the three channels of RGB towards the color of the facial blood flow, we can combine the three channels: the three channels contain the same heart rate information, the intensity of other interference signals is different in each channel; the three channels of RGB can be combined in the frequency domain according to the Fourier transform, and the time domain information after the combination is obtained by the inverse Fourier transform, as shown in FIG. 4, and the periodic signal is a heart rate signal, and the envelope is a blood signal.

The facial dimension reduction image obtained through the camera includes three channels of R, G, and B (red, green, blue); since the differences of the wavelengths of the three colored lights, the depth of the penetration into the skin is also different, which reflects different information: the red reflects more accurate blood flow information due to a large penetration depth, and also contains a lot of noise information such as muscle activity; the green is considered as the most common heart rate detection light, obtains the most stable blood flow information(anti-motion interference, anti-physiological noise and the like); the blue is the least light-permeable light, most effective in anti-motion interference. Combining the signals of the three channels can effectively amplify and extract the heart rate/blood flow information.

Methods of channel merging include, but are not limited to, light channel projection, entropy calculation, and the like.

Entropy calculation: the main idea of channel entropy merging is to obtain the probability specific gravity of each channel by performing signal transformation on the three-channel information.

Light channel projection: the signals obtained by combining the channels are the original blood flow signals, and the signals within the effective heart rate range can be extracted through bandpass filtering (0.6-3 Hz). Further, the heartbeat frequency can be calculated by means of a peak detection in the time domain or Fourier transform in frequency and the like. In some embodiment, the filtered signal is subjected to a fast Fourier transform, and the obtained frequency-domain peak is tracked and the most likely heart rate frequency is identified; if the maximum peak value exceeds 2 times the second peak value, the confidence level of the heart rate calculation can be considered high; if the maximum peak value of the heart rate is continuously lower than a certain threshold value, the detection is considered unstable and the user can be reminded to improve the index parameters such as the ambient light.

After the heart rate is calculated, the effective heart rate with a high confidence index can be further selected in combination with the confidence index, and the long-term heart rate trend of the user can be displayed at the user terminal according to the calculation result of the heart rate. If the continuous heart rate is further analyzed, high-level parameters such as tension, cardiac health, heart rate variation and the like can be obtained.

The combination of the face recognition algorithm and the facial heart rate algorithm makes it more effective to display detailed information at the user end when the person's been working: the time period during which the person appears in front of the workbench, and the change rule of the heart rate during the corresponding period. When the heart rate is relatively higher, it can remind the user to relax and exercise.

Finally, it should be noted that the above embodiments are merely illustrative of the technical solution of the present invention and are not to be limited thereto; although the invention has been described in detail with reference to the foregoing embodiments, it will be understood by those skilled in the art that they may still be modified or substituted for some of the technical features described above without departing from the spirit and scope of the embodiments of the present invention and without materially departing from the spirit and scope of the embodiments of the invention.

I claim:

1. A human body physiological parameter monitoring method based on face recognition for a workstation, the method is based on a human physiological parameter monitoring system; the said human physiological parameter monitoring system comprises a backstage server, at least one image acquisition device arranged in the workstation; the image acquisition device is communicatively connected with the backstage server, wherein the method comprises the following steps:
(1) continuous image sampling is carried out by the image acquisition device, and uploaded to the backstage server; when an image acquisition device detects the presence of a person, it proceeds to step (2);
(2) the backstage server compares the person detected in step (1) with a pre-stored registered person sample on the backstage server through a face recognition algorithm;
if the current person is a registered object, the backstage server stores the person's current physiological parameter information into a database for this person for subsequent analysis;
if the current person is an unregistered object, the person is ignored.

2. The human body physiological parameter monitoring method based on face recognition for a workstation of claim 1, wherein it further comprises a user terminal device communicatively connected with the backstage server, and the current working status of the system comprises a registering status and a monitoring status; the current working status of the system is initialized to the monitoring status upon power-on, and the user terminal device communicates with the backstage server through a pre-agreed communication mode to enter the registering status; in the said step (1), before the continuous image sampling is performed by the image acquisition device in the workstation and uploaded to the background server, the backstage server judges whether the current working status of the system is a registering status or a monitoring status, and only when the current working status of the system is the monitoring status, continuous image sampling is performed by the image acquisition device in the workstation and uploaded to the backstage server, otherwise, it proceeds to step (3): the current working status of the system is the registering status, and the head portrait of the person is stored in the database for this person.

3. The human body physiological parameter monitoring method based on face recognition for a workstation of claim 2, wherein the user terminal device is a mobile phone; when the current working status of the system is the registering status, the head portrait is collected by the camera of the mobile phone and uploaded to the backstage server, and the backstage server saves the head portrait of the person to the database for this person.

4. The human body physiological parameter monitoring method based on face recognition for a workstation of claim 1, wherein the physiological parameter information of a person includes heart rate and blood flow, and the heart rate and blood flow are obtained by analyzing the continuous frame images acquired by the image acquisition device.

5. The human body physiological parameter monitoring method based on face recognition for a workstation of claim 4, wherein it comprises the following steps to obtain the heart rate and blood flow information through analyzing the continuous frame images acquired by the image acquisition device:
S1, capture continuous frame images of a person;
S2, extract the collected RGB information of the skin area of each frame image, and then obtain three matrices based on the information of the three channels of the extracted RGB;
S3, perform a dimension reduction on the three matrices obtained in each frame image in step S2, and three new matrices are obtained in each frame image;
S4, average the three new matrices obtained in each frame image in step S3, and respectively obtain an average value of each new matrix of each frame image; then use “time” as the abscissa, and “the average value of the new matrix of the R channel” as the vertical ordinate to obtain a first waveform diagram; use “time” as the abscissa, and “the average value of the new matrix of the G channel” as the vertical ordinate to obtain a second waveform diagram; use “time” as the abscissa, and “the average value of the new matrix of the B channel” as the vertical ordinate to obtain a third waveform diagram;
S5, filter the three waveform diagrams obtained in step S4 through a filter;
S6, combine the three waveform diagrams filtered in step S5;
S7, extract the periodic signal as a heart rate signal and the envelope signal as a blood flow signal in the waveform diagram combined in step S6.

6. The human body physiological parameter monitoring method based on face recognition for a workstation of claim 5, wherein step Si is acquiring a face image, and step S2 is extracting facial skin region.

7. The human body physiological parameter monitoring method based on face recognition for a workstation of claim 5, wherein in step S4, the three new matrices need to be subjected to weighted average calculation; the weighted average calculation method is: sequentially arranging the difference values of frames before and after the dimension reduction matrix, filtering out pixels whose absolute value of change is greater than a set threshold value, calculating the average value of the remaining pixel values and this value is the average value of the channel of the current frame.

8. The human body physiological parameter monitoring method based on face recognition for a workstation of claim 5, wherein the dimension reduction in step S3 refers to smoothing and downsizing the matrix.

9. The human body physiological parameter monitoring method based on face recognition for a workstation of claim 5, wherein before acquisition in step S1, image brightness detection is further required, and if the detected image brightness is insufficient, exposure compensation is required until the detected image brightness meets the standard.
